(19) United States
(12) Patent Application Publication
(10) Pub. No.: US 2006/0223139 A1
(43) Pub. Date: Oct. 5, 2006

(54) Use of fluorinated fluids as storage liquid for preserved biological specimens

(76) Inventor: David A. Hesselroth, Hudson, WI (US)

Correspondence Address: 3M INNOVATIVE PROPERTIES COMPANY, PO BOX 33427, ST. PAUL, MN, 55133-3427, US

(21) Appl. No.: 11/385,372

(22) Filed: Mar. 21, 2006

Related U.S. Application Data
(60) Provisional application No. 60/663,782, filed on Mar. 21, 2005.

(51) Int. Cl. G01N 1/30; G01N 33/48

(52) U.S. Cl. 435/40.52

(57) ABSTRACT

A method of preserving a biological tissue specimen comprising at least one of fixing or dehydrating a specimen, and substantially completely immersing the specimen in a preserving fluid comprising one or more fluorinated hydrocarbons. Also, a biological tissue specimen immersed in a preserving fluid comprising one or more fluorinated hydrocarbon fluids.

### CROSS-REFERENCE TO RELATED APPLICATION

This application claims priority to U.S. Provisional Patent Application No. 60/663,782, filed Mar. 21, 2005.

### FIELD OF INVENTION

This invention relates to the preservation of biological specimens, e.g., animal and plant tissue specimens. In particular, it relates to preservation of tissue specimens following fixation and/or dehydration.

### BACKGROUND

A number of institutions routinely keep biological tissue specimens for display and/or research purposes.

Currently biological tissue specimens are routinely preserved with fixatives to preserve the specimens for storage and subsequent display and/or some forms of later examination. The particular fixative approach is chosen based on the nature of the sample and examination(s) for which the specimen is being prepared. In performing their protective role, fixatives denature proteins by coagulation, by forming additive compounds, or by a combination of the two, such that the proteins in the tissue specimen are stabilized and the cellular structure of the specimen is protected.

One common fixative approach is to use formaldehyde or formaldehyde-based fixative compositions such as formalin (e.g., aqueous solutions containing effective amounts of formaldehyde, e.g., about 10% w/w in water). The specimen is immersed in the fixative composition for a period of time, typically several days or even weeks depending upon the specimen, during which time the formaldehyde reacts with the specimen to form crosslinks, e.g., the basic amino acids to form “methylene bridges” that prevent breakdown of tissues resulting from release of enzymes after tissue death (“autolysis”). The specimen may then be rinsed in succeeding baths of alcohol (e.g., ethyl alcohol or isopropyl alcohol) to remove the residual formaldehyde. Finally, the sample is typically retained in an alcohol solution, e.g., 70% w/w ethyl alcohol and 30% w/w water, 100% w/w methanol, or 80% w/w propanol. In some instances, the specimen is maintained indefinitely in a formaldehyde-based composition, e.g., formalin.

In some instances, tissue specimens are preserved by dehydrating, e.g., with rinses of water-extracting fluids such as alcohols. Following dehydration, such specimens are commonly stored in alcohol-based solutions to preserve them. U.S. Pat. No. 4,911,915 discloses such a technique using a series of blends of methyl alcohol and isopropyl alcohol.

Many institutions such as universities, museums, and medical facilities possess inventories containing thousands or even millions of samples. The use of formaldehyde or formaldehyde-based fixative compositions is of concern because of certain health risks associated with such materials. The use of alcohols for preparation and/or storage of specimens is problematic as the flammable material poses a safety hazard and requires special facilities for safe storage.

### SUMMARY OF INVENTION

The present invention provides novel approaches for preservation and storage of tissue samples or specimens and tissue specimens that have been preserved in such fashion.

In brief summary, the method of the invention comprises: (a) providing a tissue specimen, (b) preparing the specimen by at least one of fixing or dehydrating the specimen, and (c) substantially completely immersing the specimen in a preserving fluid as described herein. In some embodiments, the present invention can be used to prepare and preserve newly taken specimens. In other embodiments, the present invention can be used to preserve tissue specimens that were previously prepared and preserved using conventional techniques and which are currently preserved in formaldehyde or formaldehyde-based fluids, or which are currently preserved in alcohol-based fluids.

Briefly summarizing, tissue specimens of the invention comprise tissue samples that are substantially completely immersed in a preserving fluid as described herein.

The present invention provides a number of significant advantages in the process for preserving tissue specimens as well as the preservation of such specimens. Importantly, excellent preservation performance can be obtained. Preservation fluids of the invention tend remain clear, making the thusly preserved specimens well-suited for display, e.g., in museums, class rooms, etc. Preservation compositions of the invention are non-flammable. Thus, preserved specimens are safer to transport, store, and work with. Also, when formaldehyde or formaldehyde-based compounds such as formalin are replaced, the toxicity and risk of using a suspected cancer-causing agent is eliminated.

### DETAILED DESCRIPTION OF ILLUSTRATIVE EMBODIMENTS

In brief summary, the method of the invention comprises: (a) providing a tissue specimen, (b) preparing the specimen by at least one of fixing or dehydrating the specimen, and (c) substantially completely immersing the specimen in a preserving fluid as described herein.

The present invention may be used to preserve a variety of plant or animal tissue specimens. Illustrative examples of animal tissue that may be preserved with the present invention include muscle, organ, fat, skin, bone, collagen, and connective tissue. The invention may similarly be used with other animal tissue specimens as well as a variety of plant tissue specimens.

The tissue specimen is prepared for preservation by at least one of fixing or dehydrating. As will be understood by those skilled in the art, selection of the manner of preparation will be dependent in part upon the purposes for which the specimen is being preserved and availability of preparation materials and equipment. For example, specimens which are intended to be used for display purposes, e.g., in museums and class rooms, will typically be prepared by fixing, whereas samples which are intended to be used for analytical purposes, e.g., examination under microscope, etc., will typically be prepared by dehydrating.

In some embodiments of the invention, the tissue specimen will be prepared by fixing with a fixing agent selected from the group consisting of formaldehyde and blends of formaldehyde with other fluids in convention fashion.

Typically, fixing comprises substantially completely immersing the specimen in the fixing agent for a sufficient period of time to preserve the tissue, typically a minimum of 48 hours is used. The specimen being preserved must be prepared in way that the fixing agent will permeate into all portions of the tissue before significant decomposition begins.

Common fixing agents that may be used include formaldehyde or formaldehyde-based fixative compositions such as formalin (e.g., aqueous solutions containing effective amounts of formaldehyde, e.g., about 10% w/w in water). The specimen is immersed in the fixing agent composition for several days or even weeks depending upon the specimen, during which time the formaldehyde reacts with the specimen to form crosslinks, e.g., the basic amino acids to form “methylene bridges” that prevent breakdown of tissues resulting from release of enzymes after tissue death (“autolysis”). The specimen may then optionally be rinsed in succeeding baths of alcohol (e.g., ethyl alcohol or isopropyl alcohol) to remove the residual formaldehyde.

Following such fixing, and the optional alcohol rinse, the specimen is immersed, preferably substantially completely, in a preserving fluid as discussed below.

In some embodiments, the specimen will be prepared by dehydrating. One method of dehydration well known in the art comprises comprises treating the specimen with water-extracting liquids to extract water therefrom. Typically, the specimen will be contacted with, e.g., rinsed with or substantially completely immersed in one or more baths of suitable water-extracting liquid. Illustrative examples of compositions used as dehydrants include compositions comprising one or more alcohols.

After it is prepared, i.e., treated by fixing and/or dehydrating, the specimen is preserved by immersing it, preferably substantially completely, in fluorinated hydrocarbon fluid.

The specimen is held, preferably substantially completely, covered by the preserving fluid. This might be accomplished by placing the specimen in vessel and submerging it completely with preserving fluids. Preferably, the vessel will be sealed to prevent evaporation of preserving fluid which might lead to exposure of the specimen to the atmosphere and thus fail to achieve desired preservation. The specific density of some of the preserving fluids which may be used in accordance with the invention is relatively high, thus it may be necessary to take steps to ensure that the sample is substantially completely immersed in the preserving fluid, e.g., evacuate the vessel before sealing it, placing a wicking wrap around the specimen, completely filling the portion of the vessel in which the specimen is held with preserving fluid, etc.

Preserving fluids of the invention comprise one or more fluorinated hydrocarbon fluids that are preferably normally liquid at room temperature and pressure. The fluorinated hydrocarbon fluid may be partially fluorinated or fully fluorinated, i.e., perfluorinated. The fluid may contain some other halogens, e.g., chlorine or bromine. Illustrative examples of fluorinated hydrocarbon fluids that may be useful herein are selected from the group consisting of hydrofluoroethers (“HFEs”), e.g., NOVEC™ Engineered Fluids from 3M Company; (“HCFCs”) hydrochlorofluorocarbons such as ASAHIKLIN™ AK-225 from Asahi Glass and HCFC-141b from DuPont; hydrofluorocarbons (“HFCs”) such as VERTREL™ XF from DuPont; perfluorocarbons (“PFCs”) such as 3M™ Fluoroinert™ Liquids; and chlorofluorocarbons (“CFCs”). HFEs are typically preferred as they provide excellent performance in accordance with the invention, are safe to work with, and exhibit low global warming potential. In some embodiments, the preserving fluid will consist essentially of one or more fluorinated hydrocarbon fluids.

In some other embodiments, the preserving fluid will further comprise one or more co-mediums. Selection of the type(s) and amount(s) of co-medium will be dependent in part upon the nature of the specimen being preserved. Those with ordinary skill in the art will be able to readily determine whether certain co-mediums may be used for preservation of particular specimens. Illustrative examples of co-mediums which may be used include alcohols, hydrocarbons, or other common organic solvents.

The preserving fluid is preferably nonflammable. By “nonflammable” it is meant that the preserving fluid has no flashpoint and can not sustain a flame.

Preferably, the preserving fluid is substantially immiscible with water, accordingly the preserving fluid will not extract water from any residual fixing agent or the tissue sample after preparation, making it's preserving properties more stable.

The preserving fluid is substantially insoluble with formaldehyde and vice versa, accordingly any formaldehyde will remain in place as a crosslinking fixative, enhancing the stability and appearance of tissue sample samples that have been prepared by fixing with formaldehyde-based materials.

Preserved specimens that have been previously prepared and stored in alcohol, either with formaldehyde or formalin fixation or via alcohol fixation, can be preserved in reservoirs of preserving fluids in accordance with the present invention. Replacing alcohol as the storage medium makes such specimens safer to transport, store, and work with by reducing the potential hazards of fire. In addition, the preserving fluids described herein may present other safety and performance advantages as compared to previously known alcohol-containing fixatives and storage media.

Briefly summarizing, tissue specimens of the invention comprise tissue samples that are substantially completely immersed in a preserving fluid as described herein.

### EXAMPLES

The invention will be further explained by the following illustrative examples but the particular materials and amounts thereof recited in these examples, as well as other conditions and details, should not be construed to unduly limit this invention.

Unless otherwise indicated, all amounts are expressed in parts by weight.

Test Fluids

| Designator | Name | Availability |
|---|---|---|
| HFE 7100 | 3M ™ NOVEC ™ Engineered | 3M Company, St. Paul |
| | Fluid HFE-7100 | MN |
| HFE 7200 | 3M ™ NOVEC ™ Engineered | 3M Company, St. Paul |
| | Fluid HFE-7200 | MN |
| FC-77 | 3M ™ FLUORINERT ™ | 3M Company, St. Paul |
| | Electronic Fluid FC-77 | MN |
| HFE- | 3M ™ NOVEC ™ Engineered | 3M Company, St. Paul |
| 71IPA | Fluid HFE-71IPA | MN |
| CFC-113 | trichlorotrifluoroethane | DuPont, Wilmington, DE |
| VERTREL | Dupont ™ VERTREL ™ XF | DuPont, Wilmington, DE |
| XF | Specialty Fluid | |
| HCFC-225 | ASAHIKLIN ™ AK-225 | AGC Chemicals |
| | | Americas, Inc., Bayonne, |
| | | NJ |

Unless otherwise indicated, the following test methods were used.

Control Samples

Six worms (Lumbricus terrestris) were euthanized in ethanol, cut open and cleaned with a 10% formaldehyde/water (w/w) solution then placed in a 100 ml jar containing 10% formaldehyde solution. Portions of the worm tissue were removed after 48 hours for testing with other test fluids as described below. Additionally, portions of the worm tissue were removed after 1 week and 10 weeks for the purpose of creating microscopic sections. Slides were prepared, stained with hematoxylin and eosin and examined under a light microscope. The appearance of the tissue preserved in 10% formaldehyde showed excellent muscle feathering and no sign of degradation or decomposition after 1 and 10 weeks

Another six worms (Lumbricus terrestris) were euthanized in ethanol, cut open and cleaned with a 70% ethanol/water (w/w) solution then placed in a 100 ml jar containing 70% ethanol/water. Portions of the worm tissue were removed after 48 hours for testing with other test fluids as described below. Additionally, portions of the worm tissue were removed after 1 week and 10 weeks for the purpose of creating microscopic sections. Slides were prepared, stained with hematoxylin and eosin and examined under a light microscope. The appearance of the tissue preserved in 70% ethanol showed excellent muscle feathering and no sign of degradation or decomposition after 1 and 10 weeks.

### Example 1

A portion of the worm tissue was removed from the 10% formaldehyde solution after 48 hours. The tissue was placed in a 100 ml jar containing 3M™ NOVEC™ Engineered Fluid HFE-7100 and the container was sealed with a lid. Portions of the tissue were removed after 1 week and 10 weeks and processed for the purpose of creating microscopic sections. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 2

The procedure described in Example 1 was followed using 3M™ NOVEC™ Engineered Fluid HFE-7200. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 3

The procedure described in Example 1 was followed using 3M™ NOVEC™ Engineered Fluid HFE-71IPA. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 4

A portion of the worm tissue was removed from the 10% formaldehyde solution after 48 hours. Water was removed from the tissue sample by sequential rinsing in solutions comprising 70%, 80%, 95% and 100% ethanol. The dehydrated tissue was placed in a 100 ml jar containing HFE-7100 and the container was sealed with a lid. Portions of the tissue were removed after 1 week and 10 weeks and processed for the purpose of creating microscopic sections. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 5

The procedure described in Example 4 was followed using HFE-7200. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 6

The procedure described in Example 4 was followed using HFE-71IPA. Slides were prepared, stained with hematoxylin and eosin and examined under a light microscope. The appearance of the tissue at both 1 and 10 weeks showed excellent muscle feathering and looked no different than tissue samples stored in the 10% formaldehyde.

### Example 7

A portion of the worm tissue was removed from the 70% ethanol solution after 48 hours. Water was removed from the tissue sample by sequential rinsing in solutions comprising 70%, 80%, 95% and 100% ethanol. The dehydrated tissue was placed in a 100 ml jar containing HFE-7100 and the container was sealed with a lid. Portions of the tissue were removed after 1 week and 10 weeks and processed for the purpose of creating microscopic sections. Slides were prepared, stained with hematoxylin and eosin and examined under a light microscope. The appearance of the tissue at both 1 and 10 weeks showed excellent muscle feathering and looked no different than tissue samples stored in the 70% ethanol.

### Example 8

The procedure described in Example 7 was followed using HFE-7200. Slides were prepared, stained with hematoxylin and eosin and examined under a light microscope. The appearance of the tissue at both 1 and 10 weeks showed excellent muscle feathering and looked no different than tissue samples stored in the 70% ethanol.

### Example 9

The procedure described in Example 7 was followed using HFE-71IPA. Slides were prepared, stained with hematoxylin and eosin and examined under a light microscope. The appearance of the tissue at both 1 and 10 weeks showed excellent muscle feathering and looked no different than tissue samples stored in the 70% ethanol.

### Comparative Example 1

A portion of the worm tissue was removed from the 70% ethanol solution after 48 hours. The tissue was placed in a 100 ml jar containing HFE-7100 and the container was sealed with a lid. Portions of the tissue were removed after 1 week and 10 weeks and processed for the purpose of creating microscopic sections. Slides were prepared, stained with hematoxylin and eosin and examined under a light microscope. After one week the tissue looked no different than tissue samples stored for a week in 70% ethanol however after 10 weeks the samples showed significant cell degradation consistent with decomposition. There was no observable muscle feathering or cohesiveness. This comparative example demonstrates the need to prepare the specimen, e.g., by fixing or dehydrating it, before placing it in the preserving fluid.

### Comparative Example 2

The procedure described in Comparative Example 1 was followed using HFE-7200. Slides were prepared, stained with hematoxylin and eosin and examined under a light microscope. After one week the tissue looked no different than tissue samples stored for a week in 70% ethanol however after 10 weeks the samples showed significant cell degradation consistent with decomposition. There was no observable muscle feathering or cohesiveness. This comparative example demonstrates the need to prepare the specimen, e.g., by fixing or dehydrating it, before placing it in the preserving fluid.

### Comparative Example 3

The procedure described in Comparative Example 1 was followed using HFE-71IPA. Slides were prepared, stained with hematoxylin and eosin and examined under a light microscope. After one week the tissue looked no different than tissue samples stored for a week in 70% ethanol however after 10 weeks the samples showed significant cell degradation consistent with decomposition. There was no observable muscle feathering or cohesiveness. This comparative example demonstrates the need to prepare the specimen, e.g., by fixing or dehydrating it, before placing it in the preserving fluid.

### Examples 10-13

Five worms (Lumbricus terrestris) were euthanized in ethanol, cut open and cleaned with a 10% formaldehyde/water (w/w) solution. The cleaned worms were then placed in a 100 ml jar containing 10% formaldehyde for 48 hours and then transferred to 100 ml jars containing a variety of test fluids. The jars were sealed and visual examination of the worms was made over a period of 8 weeks. Samples that showed clear fluid and no change in tissue appearance were rated “good”. The results are shown in Table 1 below.

TABLE 1

| | | Visual Specimen Quality | | | | | |
|---|---|---|---|---|---|---|---|
| Example | Test Fluid | 1 week | 2 weeks | 4 weeks | 5 weeks | 6 weeks | 8 weeks |
| 10 | FC77 | good | good | good | good | good | good |
| 11 | HCFC 225 | good | good | good | good | good | good |
| 12 | CFC-113 | good | good | good | good | good | good |
| 13 | VERTREL XF | good | good | good | good | good | good |

Various modifications and alterations of this invention will become apparent to those skilled in the art without departing from the scope and spirit of this invention.

What is claimed is:

1. A method of preserving a biological specimen comprising: (a) providing a tissue specimen, (b) at least one of fixing or dehydrating said specimen, and (c) substantially completely immersing said specimen in a preserving fluid wherein said preserving fluid comprises one or more fluorinated hydrocarbons.

2. The method of claim 1 wherein said fluorinated hydrocarbon is selected from partially fluorinated hydrocarbons and perfluorinated hydrocarbons.

3. The method of claim 2 wherein said fluorinated hydrocarbon is selected from the group consisting of hydrofluoroethers, hydrochlorofluorocarbons, perfluorocarbons, hydrofluorocarbons, chlorofluorocarbons, and blends thereof.

4. The method of claim 1 wherein said preserving fluid further comprises a co-medium.

5. The method of claim 1 wherein said preserving fluid is nonflammable.

6. The method of claim 1 wherein said fixing comprises treating said specimen with a fixing agent selected from the group consisting of formaldehyde and blends of formaldehyde with other fluids.

7. The method of claim 6 wherein said fixing comprises substantially completely immersing said specimen in said fixing agent for a sufficient period of time to substantially completely permeate said specimen.

8. The method of claim 1 wherein said dehydrating comprises treating said specimen with water-extracting liquids to extract water therefrom.

9. The method of claim 8 wherein said water-extracting liquid is a composition comprising one or more alcohols.

10. The method of claim 8 wherein said treating comprises applying said water-extracting liquid to said specimen in two or more rinses.

11. A biological specimen immersed in a preserving fluid comprising one or more fluorinated hydrocarbon fluids.

12. The specimen of claim 11 wherein said specimen was fixed prior to immersion in said preserving fluid.

13. The specimen of claim 11 wherein said specimen was dehydrated prior to immersion in said preserving fluid.
